(19) United States
(12) Patent Application Publication
(10) Pub. No.: US 2011/0297157 A1
(43) Pub. Date: Dec. 8, 2011

(54) RESPIRATION SYSTEM

(75) Inventor: Oliver WALLNEWITZ, Lubeck (DE)

(73) Assignee: DRAGER MEDICAL GMBH, Lubeck (DE)

(21) Appl. No.: 13/150,623

(22) Filed: Jun. 1, 2011

(30) Foreign Application Priority Data
Jun. 2, 2010 (DE) 10 2010 022 545.2

(51) Int. Cl. A61M 16/06

(52) U.S. Cl. 128/206.21

(57) ABSTRACT

A respiration system with a breathing mask, a connection device formed on the breathing mask for connecting the breathing mask to a flexible air tube, with a device for attaching the breathing mask on the head of a patient with a contact unit for being placed on the head of the patient and with at least one connecting unit for connecting the contact unit to the breathing mask. The respiration system also includes at least one load-sensing device for displaying the tensile force occurring in the at least one connecting unit.

### CROSS REFERENCE TO RELATED APPLICATIONS

This application claims the benefit of priority under 35 U.S.C. §119 of German Patent Application DE 10 2010 022 545.2 filed Jun. 2, 2010, the entire contents of which are incorporated herein by reference.

### FIELD OF THE INVENTION

The present invention pertains to a respiration system (also known as a ventilation system) with a breathing mask, a connection means formed on the breathing mask for connecting the breathing mask to a flexible air tube, a means for attaching the breathing mask to the head of a patient with a contact unit for being placed on the head of the patient and with at least one connecting unit for connecting the contact unit to the breathing mask.

### BACKGROUND OF THE INVENTION

Respiration systems are used for noninvasive respiration (also known as ventilation) of patients. Respiration systems comprise a breathing mask that is placed on the face area of the patient and generally covers both the nose and mouth, so that the patient can be supplied with artificial breathing air when breathing through the nose and mouth.

The breathing mask is generally attached on the face area of the patient by means of an elastic strap acting as a connecting unit. For example, a contact cloth or a net acting as a contact unit, which lies on the back of the patient's head, so that the breathing mask is pressed as a result onto the face area of the patient because of an elastic prestress of the elastic straps, is attached to the strap. If the pressing force applied by the straps onto the face area of the patient is too weak, leaks may occur, especially in the area of the bridge of the nose or the area around the eyes. As a result, artificial respiration is no longer possible in a sufficiently effective manner. If the force with which the breathing mask is pressed onto the face area of the patient is too strong, injury, for example, decubitus of the bridge of the nose and consequently necroses may develop, especially during prolonged and frequent use. Neither development of leaks and consequently ineffective artificial respiration of the patient nor injuries because of an excessive pressing force of the breathing mask on the face area of the patient is desired during the use of the respiration system with breathing mask.

DE 103 38 169 A1 shows a respiration system which comprises a forehead support arranged on the breathing mask. The forehead support can be attached in various positions through a slot provided with catches. The breathing mask is attached to the head of a patient to be respirated with a headband and a neckband.

DE 1 251 160 shows a respirator half mask with arms articulated laterally to the mask body, which are connected to a connection piece supported at the back of the head, the arms being articulated to the mask body such that they can be folded against this mask body and having elastic means which automatically pivot into the use position. The holding means of the mask body are designed as elastically flexible holding means.

U.S. Pat. No. 3,850,168 shows a respiration system with a breathing mask. A contact unit lies on the back of the patient's head. The contact unit is connected to the breathing mask by means of a connecting unit, so that the breathing mask is pressed as a result onto the face area of the patient with a pressing force. Springs are incorporated in the two connecting units, so that the breathing mask is pressed onto the face area with a pressing force because of an elastic prestress of the springs. The length of the connecting unit can be adjusted by means of adjusting units and the force applied by the springs onto the breathing mask is thus also changed for a different pressing force of the breathing mask on the face area of the patient.

### SUMMARY OF THE INVENTION

An object of the present invention is to make available a respiration system in which injuries in the face area of the patient because of an excessive pressing force of the breathing mask can be reliably avoided. The respiration system shall, furthermore, be able to be handled simply and reliably and have low manufacturing costs.

This object is accomplished with a respiration system, comprising a breathing mask, a connection means formed on the breathing mask for connecting the breathing mask to a flexible air tube, a means for attaching the breathing mask to the head of a patient with a contact unit for being placed on the head of the patient and with at least one connecting unit for connecting the contact unit to the breathing mask, wherein the respiration system comprises at least one load-sensing device for displaying the tensile force occurring in the at least one connecting unit.

The pressing force applied by the breathing mask on the face area of the patient corresponds approximately to the sum of the tensile force occurring in the at least one connecting unit. The tensile force occurring can be displayed by means of the load-sensing device in at least one connecting unit, so that an attending physician can recognize whether the breathing mask lies on the face area of the patient with an excessively strong, excessively weak or essentially correct pressing force. The development of excessive leaks at the breathing mask or injuries in the face area of the patient due to an excessive pressing force, especially in the area of the nose, can be avoided as a result.

The at least one load-sensing device is preferably incorporated in the at least one connecting unit and/or in the breathing mask. The load-sensing device can be incorporated in the connecting unit and/or in the mask, for example, by means of fusing together, bonding or ultrasonic welding of the connecting unit and/or mask with the load-sensing device. The connecting unit and mask can be connected in one piece especially in this way by means of the load-sensing device.

Between the connecting unit and breathing mask may be arranged a forehead support, by means of which a reduction of the pressure on the bridge of the nose is brought about. Of course, the load-sensing device may also be incorporated in the forehead support in this case.

In another embodiment, the at least one load-sensing device is incorporated in an attaching means, which makes possible a detachable connection between the connecting unit and mask. The at least one attaching means is preferably part of the breathing mask, i.e., it may be made in one piece with the mask or be connected to the mask by means of fusing, bonding or ultrasonic welding, so that the load-sensing device is incorporated in the mask by means of the attaching means. The connecting unit is then attached to the attaching means, for example, by means of a Velcro connection. Of course, the attaching means and mask may also be connected, for example, by means of a clevis type eyelet or a Velcro fastener. In this case, the connecting unit can be connected to the attaching element both in one piece and, for example, by means of a Velcro connection. The at least one load-sensing device is preferably incorporated in the at least one connecting unit and/or in the breathing mask and/or in the forehead support.

In an additional embodiment, at least one elastic element is integrated in the at least one connecting unit; in particular, the at least one connecting unit is embodied as the elastic element. The connecting unit is embodied here, for example, as an elastic strap.

In an additional variant, the at least one load-sensing device is a spring with a scale, e.g., a color scale or numeric scale. As an alternative or in addition hereto, the at least one load-sensing device is a wire strain gauge, whose color is preferably variable as a function of the tensile force.

In an additional embodiment, the at least one load-sensing device is an electric and/or electronic load-sensing device and the load-sensing device preferably comprises a piezo element. It is thus possible that an acoustic display can also be generated, besides an optical display, in case the tensile force in the connecting unit exceeds or drops below a preset tensile force. The at least one load-sensing device preferably also comprises an energy supply unit, for example, a battery.

The respiration system preferably comprises an upper connecting unit and lateral connecting units.

In an additional embodiment, a load-sensing device detects the tensile force occurring in the upper connecting unit.

In another embodiment, only one load-sensing device is arranged, and it is arranged at the upper connecting unit only.

In an additional embodiment, the at least one connecting unit is a preferably elastic strap. The contact unit is a headband or a preferably textile contact cloth or a net for lying on the back of the head of a patient. The at least one attaching means is a clevis type eyelet or a hook and loop fastener—Velcro® fastener. The respiration system comprises at least one means for changing the pressing force of the breathing mask on the face area of a patient.

The at least one means for changing a pressing force of the breathing mask is, for example, a means for changing the length of the preferably elastic strap, so that a stronger pressing pressure develops in case of a reduction in the length of the elastic strap and vice versa.

In an additional embodiment, the at least one means for changing the pressing force of the breathing mask may also be embodied such that at least one elastic part, for example, a spring, is incorporated in the connecting unit and this at least one elastic part absorbs no force from the connecting unit in a first position of a mechanism and thus it does not contribute to a change in the length of the connecting unit and the at least one elastic part absorbs the tensile force occurring in the connecting unit in a second position of the mechanism, as a result of which there will be a reduction in the tensile force in the connecting unit because the spring is stretched hereby and the length of the connecting unit can thus increase.

The at least one strap preferably consists at least partly of a fabric, especially one made of synthetic fibers.

An exemplary embodiment of the present invention will be described in more detail below with reference to the attached drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a front view of a respiration system;

FIG. 2 is a view of a load-sensing device in a first exemplary embodiment in status 1;

FIG. 3 is a view of the load-sensing device according to FIG. 2 in status 2;

FIG. 4 is a view of the load-sensing device according to FIG. 2 in status 3;

FIG. 5 is a view of the load-sensing device in a second exemplary embodiment; and

FIG. 6 is a schematic view showing an alternative load-sensing device integrated in a connecting unit upper strap.

### DESCRIPTION OF THE PREFERRED EMBODIMENTS

Referring to the drawings in particular, a respiration system 1 is provided for use for noninvasive artificial respiration of patients. The respiration system 1 comprises a breathing mask 2 with a connection means 3 for a flexible air tube 4 for detachably fixing the flexible air tube 4 to the breathing mask 2. The breathing mask 2 may cover, based on its geometry, both the nose and mouth of a patient, i.e., it may artificially respirate the nose and mouth simultaneously.

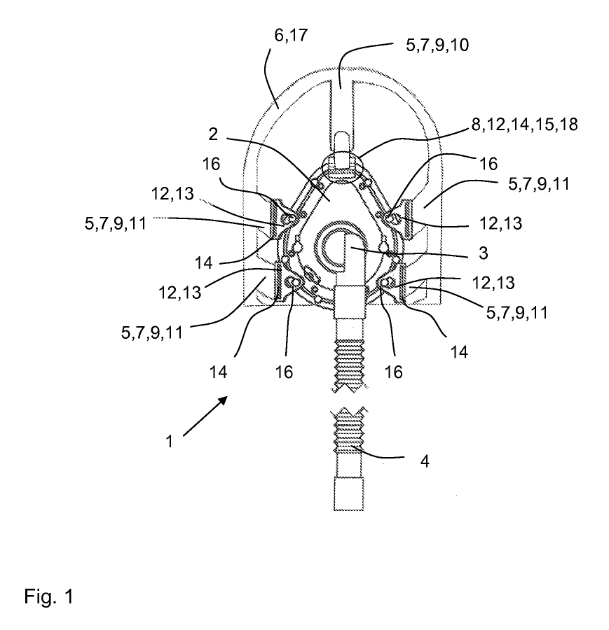

The breathing mask 2 is attached to the head, not shown, of a patient to be respirated artificially by means of an attaching means 5. The attaching means 5 comprises a contact unit 6 and a plurality of connecting units 7. The contact unit 6 is a headband 17, which lies in the area of the back of the head of the patient. The connecting units 7 connect the contact unit 6 to the breathing mask 2. The connecting units 7 comprise elastic straps 9. The straps 9 are formed of plastic and the straps comprise an upper strap 10 as well as lateral straps 11. Straps 9 are attached to the breathing mask 2 by means of attaching means 12 (FIG. 1). The attaching means 12 for the lateral straps 11 are embodied as clevis type eyelets 13 here. A suspension hook 16, which is formed on the breathing mask 2, is hooked in the clevis type eyelets 13 with a slot. As a result, the clevis type eyelets 13 can be detachably connected to the breathing mask 2 by being suspended on the suspension hooks 16. The clevis type eyelets 13 each have, in addition, a strap slot 14 each, in which the lateral straps 11 are arranged.

The upper strap 10 is likewise attached to the breathing mask 2 with the attaching means 12. The attaching means 12 for the upper strap 10 is embodied as a load-sensing device 8 according to this embodiment and is connected in one piece to the mask. The connection to the mask takes place by means of fusing the plastic of the load-sensing device with the plastic of the mask such that the attaching means 12 with the load sensing means 8 is integrated in the breathing mask 2. For example, a polycarbonate may be used as the plastic. As an alternative to the fusing, of course, other methods of attachment, such as bonding or ultrasonic welding may also be used. The upper strap 10 is attached to the load-sensing device 8 by means of a hook and loop fastener—Velcro® fastener connection and thus to the attaching means 12. For example, at the upper strap 10 there is arranged a Velcro strap 30, which is mounted at the attaching means 12 by means of a strap slot 14 and forms a Velcro connection by itself.

As an alternative to this, the load-sensing device 8 may be integrated in the connecting units 7, in particular in the upper strap 10 as shown in FIG. 6. To this end, an attaching means 12 without load-sensing device 8 can be attached to the mask, for example, by means of fusing, bonding or ultrasonic welding. The Velcro strap 30 of the upper strap 10 can be mounted and attached by means of a strap slot 14 in the attaching means 12. The load-sensing device 8 is, for example, fused, bonded or connected by means of ultrasonic welding to the plastic of the strap 10. The load-sensing device 8 has wire strain gauges 19 with a first color scale 21, a second color scale 22 and a third color scale 23.

The tensile force occurring in the upper strap 10 as a connecting unit 7, which is generated when placing the respiration system 1 on the patient, can be displayed with the load-sensing device 8.

FIGS. 2 through 4 show a first exemplary embodiment of load-sensing device 8 and attaching means 12 according to the view of the respiration system 1 in FIG. 1. The load-sensing device 8 and attaching means 12 have strap slot 14 on a strap mount 15. The upper strap 10 for attaching the load-sensing device 8 is arranged in the strap slot 14. Corresponding to the size of the patient's head, the upper strap 10 is led through the strap slot 14 and fixed to an attaching means. A hook and loop fastener—Velcro® fastener integrated in the upper strap 10 may be provided as the attaching means for this. As an alternative hereto, the upper strap 10 may be provided with a belt closure for fixing.

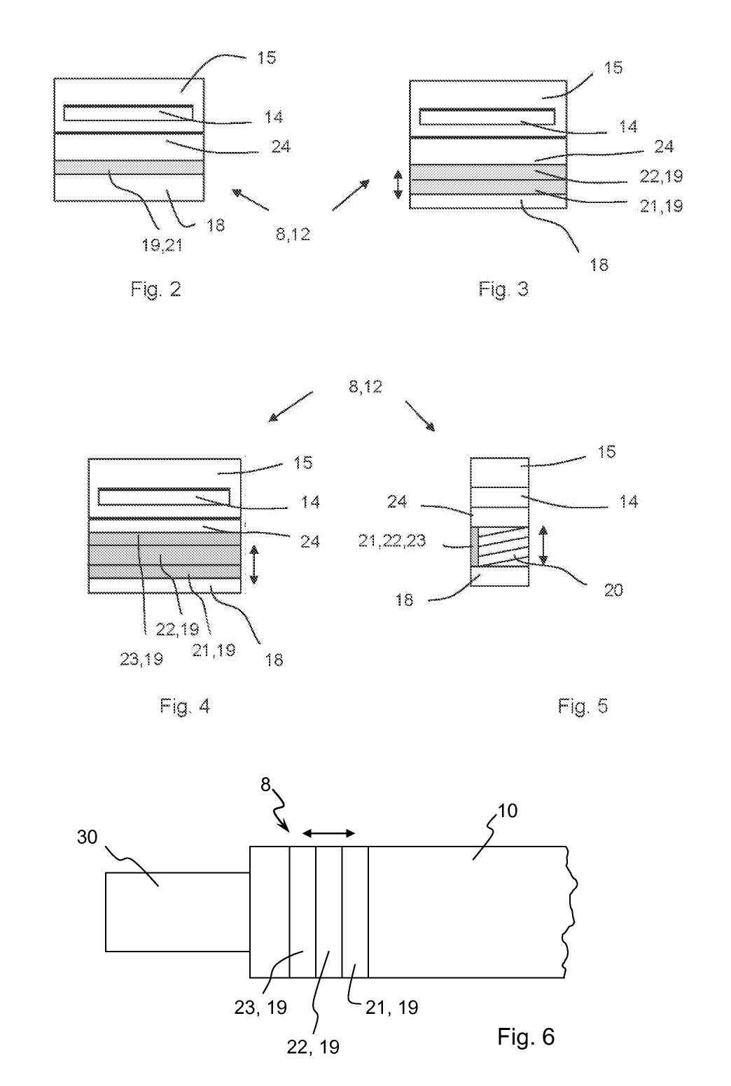

The load-sensing device 8 or attaching means 12 is attached to the breathing mask 2 by means of a mask fixing means 18 (FIGS. 2 through 4). The mask fixing means 18 may be fixed to the mask by means of fusing, bonding or ultrasonic welding. The mask fixing means 18 may be embodied, for example, as a suspension hook or as a screw connection (not shown). The load-sensing device 8 has wire strain gauges 19 with a first color scale 21 (FIG. 2), a second color scale 22 (FIG. 3) and a third color scale 23 (FIG. 4).

Only the color of the first color scale 21 is displayed in the case of the load-sensing device 8 shown in FIG. 2. The color of the first color scale 21 is green. The first color scale is displayed in a status 1. Status 1 is a range of a tensile force in the connecting unit 8 or the upper strap 10, in which the tensile force is in a range of 0 Nm to about 10 Nm.

The color of the second color scale 22 is yellow. The second color scale is displayed in a status 2. Status 2 corresponds to a tensile force in the upper strap in the range of about 10 Nm to 20 Nm.

The third color scale 23 is red. The third color scale is displayed in a status 3. Status 3 is displayed at a tensile force in the upper strap 10 if the tensile force is greater than 20 Nm.

No injury occurs and, in particular, no pressure sores are caused on the bridge of the nose in case of display of the green color of the first color scale 21 in status 1 because of the weak tensile force occurring in the face area of the patient.

The color of the wire strain gauge 19 changes from green to yellow in case of a further increase in the tensile force in the upper strap 10. A tensile force in the range of 10 Nm to 20 Nm occurs in status 2 of the load-sensing device 8. Pressure sores may develop on the bridge of the nose in the face area of the patient in this range of forces during continuous load. The attending physician thus knows when the yellow color of the second color scale 22 appears that caution is necessary and that continuous use of the breathing mask 2 in status 2 should be avoided.

The load-sensing device 8 changes over to status 3 in case of a further increase in the tensile force in the upper strap 10. The wire strain gauge 19 changes over to the third color scale 23. Injuries to the bridge of the nose develop, in general, in a short time in status 3 because of the strong pressing force of the breathing mask 2 on the face area of the patient. The attending physician can thus immediately recognize when the red color of the third color scale 23 appears that the tensile force set in the upper strap 10 is too strong and can take measures to immediately prevent injuries to the bridge of the nose.

The respiration system 1 has, besides, a force application means for changing of the pressing force of the breathing mask 2 on the face area of the patient. This force application means may be by an adjustment of a length of the upper strap 10 between the strap slot 14 and headband 17 and by changing the distance by loosening a hook and loop fastener—Velcro® fastener and subsequently moving the end of the upper strap 10 in a direction to loosen or tighten. After passing one end of the upper strap 10 through the strap slot 14, the end of the upper strap 10 is attached to the rest of the upper strap 10 by means of a Velcro® fastener. If the attending physician can recognize from the load-sensing device 8 that the tensile force in the upper strap 10 is too strong, the attending physician only needs to increase the length of the upper strap 10 between the strap slot 14 and headband 17 by loosening this Velcro® fastener and subsequently moving the end of the upper strap 10 in the direction of the load-sensing device 8, so that a weaker tensile force will occur in the upper strap 10 as a result. This also applies in the opposite sense in case of the tensile force being too weak in the upper strap 10.

FIG. 5 shows a second exemplary embodiment of the load-sensing device 8. The load-sensing device 8, which is also an attaching means 12, has a spring 20. A first color scale 21 in green, a second color scale 22 in yellow and a third color scale 23 in red are arranged at the spring 20. Because of the color scale cover 24, only one color scale 21, 22 and 23 can be seen from the outside. Because of the different position of spring 20 and hence the different length of the color scales 21, 22 and 23, three different ranges of tensile force can be displayed in the upper strap 10 analogously to the load-sensing device 8 according to FIGS. 2 through 4.

In another exemplary embodiment, not shown, the respiration system 1 also has at least one load-sensing device 8 for displaying the tensile force occurring in at least one lateral strap 11. This at least one load-sensing device 8 may be arranged alternatively or in addition to the load-sensing device 8 arranged in the upper strap (FIG. 1).

On the whole, essential advantages are associated with the respiration system 1 according to the present invention. The attending physician can visually recognize the tensile forces occurring in the upper strap 10 by means of the load-sensing device 8, so that injuries to the patient and major leaks at the breathing mask 2 can be avoided as a result because the attending physician can recognize incorrect tensile forces in the upper strap 10 and take countermeasures immediately as a result.

While specific embodiments of the invention have been shown and described in detail to illustrate the application of the principles of the invention, it will be understood that the invention may be embodied otherwise without departing from such principles.

### LIST OF REFERENCE NUMBERS

| | |
|---|---|
| 1 | Respiration system |
| 2 | Breathing mask |
| 3 | Connection means |
| 4 | Flexible air tube |
| 5 | Means for attaching the breathing mask |
| 6 | Contact unit |
| 7 | Connecting unit |
| 8 | Load-sensing device |
| 9 | Strap |
| 10 | Upper strap |
| 11 | Lateral straps |
| 12 | Attaching means |
| 13 | Clevis type eyelet |
| 14 | Strap slot |
| 15 | Strap mount |
| 16 | Suspension hook |
| 17 | Headband |
| 18 | Mask fixing means |
| 19 | Wire strain gauge |
| 20 | Spring |
| 21 | First color scale |
| 22 | Second color scale |
| 23 | Third color scale |
| 24 | Color scale cover |

What is claimed is:

1. A respiration system comprising:
a breathing mask;
a connection means formed on said breathing mask for connecting said breathing mask to a flexible air tube;
a head engaging means for engaging said breathing mask to the head of a patient, said head engaging means including a contact unit for being placed on the head of the patient and with a connecting unit for connecting said contact unit to said breathing mask; and
a load-sensing device for displaying a tensile force occurring in said connecting unit.

2. A respiration system in accordance with claim 1, wherein said load-sensing device is incorporated in said connecting unit.

3. A respiration system in accordance with claim 1, wherein said load-sensing device is integrated in said breathing mask.

4. A respiration system in accordance with claim 1, wherein:
said connecting unit includes an attachment means for attaching said head engaging means to said breathing mask; and
said load-sensing device is incorporated in said attachment means.

5. A respiration system in accordance with claim 1, wherein said connecting unit comprises an elastic element.

6. A respiration system in accordance with claim 1, wherein said connecting unit comprises an upper strap.

7. A respiration system in accordance with claim 1, wherein said connecting unit comprises lateral straps.

8. A respiration system in accordance with claim 1, wherein said connecting unit comprises an elastic strap.

9. A respiration system in accordance with claim 1, wherein said contact unit comprises at least one of a headband, a textile contact cloth and a net for being placed on a back of a patient's head.

10. A respiration system in accordance with claim 1, wherein said attaching means comprises at least one of a clevis type eyelet and a Velcro® fastener.

11. A respiration system in accordance with claim 1, further comprising at least one force application means for changing a force with which said breathing mask is pressed onto a face area of the patient.

12. A respiration system in accordance with claim 1, wherein said load-sensing device comprises a spring with a scale.

13. A respiration system in accordance with claim 12, wherein said scale is a color scale.

14. A respiration system in accordance with claim 12, wherein said scale is a numeric scale.

15. A respiration system in accordance with claim 1, wherein said load-sensing device comprises a wire strain gauge, said wire strain gauge having a color which is changed as a function of tensile force.

16. A respiration system comprising:
a breathing mask;
a connection interface on said breathing mask, the connection interface providing a connection between an interior of the breathing mask and a breathing gas tube;
a head engaging interface with a head contact unit for contact with a head of a patient and with a connecting unit for connecting the head contact unit to the breathing mask; and
a load-sensing device for displaying a tensile force occurring between said head engaging interface and said breathing mask.

17. A respiration system in accordance with claim 16, wherein said load-sensing device is incorporated in said connecting unit.

18. A respiration system in accordance with claim 16, wherein said load-sensing device is integrated in said breathing mask.

19. A respiration system in accordance with claim 16, wherein:
said head contact unit comprises at least one of a headband, a textile contact cloth and a net for being placed on a back of a patient's head; and
said connecting unit includes at least one of a clevis type eyelet and a Velcro® fastener.

20. A respiration system in accordance with claim 16, further comprising at least one force application device for changing a force applied between said head contact unit and said breathing mask for varying a force at which said breathing mask is pressed onto the face area of the patient.
